(19) United States
(12) Patent Application Publication
(10) Pub. No.: US 2005/0009740 A1
(43) Pub. Date: Jan. 13, 2005

(54) Anti-tumor vasculature effects of human serum albumin derivatives

(75) Inventors: Thomas E. Wagner, Greenville, SC (US); Xianzhang Yu, Mauldin, SC (US); Yanzhang Wei, Greer, SC (US)

Correspondence Address: FOLEY AND LARDNER;SUITE 500, 3000 K STREET NW, WASHINGTON, DC, 20007, US

(73) Assignee: Greenville Hospital System

(21) Appl. No.: 10/813,432

(22) Filed: Mar. 31, 2004

Related U.S. Application Data
(60) Provisional application No. 60/458,395, filed on Mar. 31, 2003.

(51) Int. Cl. A61K 38/38

(52) U.S. Cl. 514/8

(57) ABSTRACT

The invention relates to a pharmaceutical composition, methods for its use and kits comprising the pharmaceutical composition, wherein the composition comprises: (a) a carrier portion; (b) a targeting portion, wherein said targeting portion comprises a targeting peptide; and (c) an immune response triggering portion, wherein said immune response triggering portion triggers a complement mediated hyperacute immune response.

### BACKGROUND OF THE INVENTION

Xenograft hyperacute immune response (i.e., rejection) in humans occurs as a secondary response to a cellular glycosylation incompatibility with most non-human mammalian species. Alpha(1,3)galactosyl (agal) epitopes on the surface of cells of non-primate organs are the major xenoantigens responsible for hyperacute immune response in xenotransplantation. The antigen is synthesized by (α-1,3)galactosyl transferase (α-1,3-GT). Humans lack this enzyme, and their serum contains high levels of pre-existing natural antibody which recognizes agal epitopes and activates complement. The activation of complement ultimately leads to cellular lysis. Sandrin and McKenzie, Immunol. Rev. 141: 169-190 (1994).

A recent report discloses retroviral vector transfer of the α-(1,3)-GT gene into human tumor cells in an attempt to elicit a hyperacute immune response as an anti-cancer gene therapy strategy. Link et al., Anticancer Res. 18: 2301-2308 (1998).

Similarly, another recent report discloses the potential for delivery of the α-1,3-GT gene to sensitize human cells to complement attack as a gene therapy approach to cancer. Jaeger et al., Gene Ther. 6: 1073-1083 (1999). Retrovirus-mediated delivery of α-1,3-GT gene resulted in high level expression leading to serum-mediated lysis of five human cell targets, including endothelial and primary melanoma cells. The report found that lysis was specific for those cells expressing the antigen in a mixed cell population. The mechanism of cell lysis mimicked that involved in hyperacute rejection: activation of the classical complement pathway by natural antibody specific for agal.

Although the retrovirus-mediated delivery of α-1,3-GT gene to cells used in the gene therapy reports discussed above seems like a promising method for treating cancer in humans (at least in vitro), there are noteworthy drawbacks associated with gene therapy as a whole and in the use of retroviral vectors in gene therapy. First, gene therapy, as a therapeutic technology, is unreliable. Second, when retroviral vectors are used in gene therapy, it seems that there is little or no control over how many copies of the gene are integrated or where on the chromosome they insert. Since integration appears to be essentially random, the vector's genetic “payload” may become inserted within another important gene, disrupting or altering its expression. Third, targeting the retrovirus so that only tumor cells are infected is difficult. When non-tumor cells are infected by the retrovirus, it is possible that a gene may integrate within the regulatory region of a gene responsible for controlling cellular proliferation, thus putting the cell on the path towards cancerous growth. See Kmiec, American Scientist 87: 240-247 (1999). Fourth, it is highly unlikely that the retroviral vectors will be incorporated into every tumor cell. Thus, the tumor may survive and return if even a single tumor cell is left viable after gene therapy.

### SUMMARY OF THE INVENTION

The preferred embodiments of the present invention seek to develop a method, superior to the still unreliable retroviral vector transfer methodology of the α-(1,3)-GT gene into human cells (e.g., cancer cells). The method of the preferred embodiments of the present invention utilizes a hyperacute immune response as an anti-cancer therapy strategy.

The method of the preferred embodiments of the present invention are superior to other anti-cancer therapy strategies because the method selectively targets the tumor vasculature, including tumor neo-vasculature. The skilled artisan would recognize that the tumor vasculature is the life-line for a tumor. Thus, if one kills the cells that form the vasculature of a tumor, one kills the tumor as a whole.

In contrast, other strategies seek to either kill the tumor cells directly or to prevent the formation of new vasculature to the tumor (i.e., tumor neovasculature). Thus, for example, one might kill tumor cells with a cytotoxic and/or chemotherapeutic agent. But, even if one kills, e.g., 90% of a tumor, the remaining 10% of the tumor can re-grow and still pose a threat to an organism. Also, if one seeks to target only the tumor neovasculature, one might prevent the formation of new vasculature. The vasculature that originally “fed” the tumor, however, is still in place. Thus, the tumor survives.

In one aspect, the preferred embodiments of the present invention relate to a pharmaceutical composition comprising:
(a) a carrier portion;
(b) a targeting portion, wherein said targeting portion comprises a targeting peptide; and
(c) an immune response triggering portion, wherein the immune response triggering portion triggers a complement mediated hyperacute immune response. In a preferred embodiment of the present invention, the carrier portion is human serum albumin (HSA), the targeting peptide comprises asparagine-glycine-arginine (NGR) and the triggering portion is galactose-α-1,3-galactose.

In a second aspect, the preferred embodiments of the present invention relate to a method for selectively inducing a complement mediated hyperacute immune response to a target tissue comprising treating the tissue with a pharmaceutical composition comprising a carrier portion, a targeting portion and an immune response triggering portion, wherein the targeting portion binds to cells on said tissue. In a preferred embodiment of the present invention, the target tissue is the vasculature of a primary or metastatic solid tumor. In other preferred embodiments of the present invention, the tumor is a lung, colorectal, bladder, prostate, breast, renal, brain, pancreatic, head, neck or an ovarian tumor. In still another preferred embodiment, the carrier portion is HSA, the targeting portion is NGR and the triggering potion is gal-α-1,3-gal. In a preferred embodiment of the present invention, the method of administration of the composition is intravenous.

In a third aspect, the preferred embodiments of the present invention relate to a kit comprising, in a suitable container, a pharmaceutical composition comprising a carrier portion, a targeting portion and an immune response triggering portion. In a preferred embodiment of the present invention, the targeting portion and carrier portion is not an antibody or antibody fragment. In still another preferred embodiment of the present invention, the targeting portion selectively binds to tumor vasculature. In a preferred embodiment, the targeting portion is an inhibitor, a ligand, an agonist, an antagonist, or a substrate, where the targeting portion comprises a targeting peptide. In yet another preferred embodiment of the present invention, the targeting peptide comprises asparagine-glycine-arginine (NGR) and the triggering portion triggers a complement mediated hyperacute immune response. In a preferred embodiment, the triggering portion is galactose-α-1,3-galactose, the carrier portion is HSA, and the targeting portion is NGR.

### BRIEF DESCRIPTION OF THE DRAWINGS

FIG. 1 shows that HSA-gal successfully competed with HSA-FITC, indicating that the sugar group incorporation onto HSA does not interfere with its antibody binding affinity.

FIG. 2 shows that cell lysis is only observed in group 1 (maximum release) and little/no response in all the other cases (groups 2-7).

### DETAILED DESCRIPTION OF THE PREFERRED EMBODIMENTS

Pharmaceutical Compositions and Methods of Use

The preferred embodiments of the present invention provide a pharmaceutical composition comprising:
(a) a carrier portion;
(b) a targeting portion, wherein the targeting portion comprises a targeting peptide; and
(c) an immune response triggering portion, wherein the immune response triggering portion triggers a complement mediated hyperacute immune response.

The carrier portion of the composition of the preferred embodiments of the present invention can be a protein. In a preferred embodiment, the carrier portion is a serum protein. Serum proteins are preferred due to their inherent solubility in the blood. One advantage of such solubility is that a relatively high concentration of the carrier portion may be achieved in the blood. In a most preferred embodiment, the carrier portion is the protein human serum albumin (HSA).

HSA is a protein that comprises a myriad of reactive carboyxlate groups that not only contribute to its solubility in the blood, but that also makes the protein amenable to attaching the targeting peptides of the preferred embodiments of the present invention by standard carbodiimide chemistry.

The targeting peptide of the composition of the preferred embodiments of the present invention is an inhibitor, a ligand, an agonist, an antagonist, or a substrate. In a preferred embodiment, the targeting peptide comprises the tri-peptide motifs, e.g., asparagine-glycine-arginine (NGR). Such targeting peptides have been shown to specifically bind to the tumor vasculature and neovasculature.

It is contemplated that targeting peptides may be developed that will target cells that are cancerous, but are not associated with tumor vasculature or neovasculature. For example, targeting peptides may be developed that target leukemia cells.

Targeting peptides that do not comprise a tri-peptide motif are also contemplated. These targeting peptides include those described in U.S. Pat. Nos. 6,528,481; 6,491,894; 6,296,832; and 6,180,084.

Preferably, neither the carrier portion nor the targeting portion or peptide of the composition of the preferred embodiments of the present invention are an antibody or an antibody fragment. The inventors have found that antibodies and antibody fragments are not useful as carrier and/or targeting portions because, even if the antibody or antibody fragment comprises the galactose-α-1,3-galactose triggering portion (infra), a complement response is not observed. In fact, murine antibodies are known to contain the sugar galactose-α-1,3-galactose. Sandrin and McKenzie, Immunol. Rev. 141: 168-190. Yet, there are no reports known to the inventors that show that when these antibodies are introduced into a human, a complement response is observed.

Tri-peptide motifs like those of the preferred embodiments of the present invention are well known in the art. See e.g., Curnis, et al., Cancer Res. 62: 867-874 (2002) and Zitzmann, et al., Cancer Res. 62: 5139-5143 (2002). These tri-peptides can be directly attached to the carrier portion by first attaching a diamine to the carboxy terminus of the tri-peptide, thereby giving a tri-peptide derivative. Preferred diamines include, without limitation, ethylene diamine. At this point, the tri-peptide derivative contains reactive amines at both ends of the tri-peptide. The reactive amines are then attached to the carrier portion using standard carbodiimide chemistry thus giving a loop on the carrier portion comprising the tri-peptide.

An exemplary method for achieving such an attachment is EDC-mediated coupling of carboxylic acids on the carrier portion to the reactive amines on the tri-peptide ethylene diamine, wherein EDC is 1-ethyl-3-(3-dimethylaminopropyl) carbodiimide hydrochloride.

The tri-peptide motifs can also be incorporated into longer polypeptides, e.g., peptides containing from about 6 to about 20 amino acids; preferably from about 6 to about 12 amino acids; most preferably from about 6 to about 9 amino acids.

The targeting peptides of the preferred embodiments of the present invention may be synthesized by methods well known in the art (e.g., automated peptide synthesis).

The triggering portion of the pharmaceutical composition of the preferred embodiments of the present invention is galactose-α-1,3-galactose.

The preferred embodiments of the present application also provide a method for selectively inducing a complement mediated hyperacute immune response to a target tissue comprising treating the tissue with the above-mentioned pharmaceutical composition comprising a carrier portion, a targeting portion and an immune response triggering portion, wherein the targeting portion binds to cells on the tissue.

In a preferred embodiment, the target tissue is the vasculature and neovasculature of a primary or metastatic solid tumor. The method of the preferred embodiments of the present invention can be used to treat primary or metastatic solid tumors at, without limitation, the lung, colon, bladder, prostate, breast, kidney, brain, pancreas, head, neck and ovary. Again, by destroying the tumor vasculature and neovasculature, the tumor's blood supply is cut off and the tumor is destroyed.

Methods of Administration

The pharmaceutical composition of the preferred embodiments of the present invention can be administered as such to a human patient along with suitable carriers or excipients. Techniques for formulation and administration of drugs may be found in “Remington's Pharmaceutical Science, 17^{th} Ed., (Alfonso Gennaro, ed.) (1985).

As used herein, “administer” or “administration” refers to the delivery of a pharmaceutical composition of the preferred embodiments of the present invention to an organism in need thereof.

Suitable routes of administration include, without limitation, intravenous (IV) injections.

Alternatively, one may administer the pharmaceutical composition of the preferred embodiments of the present invention in a local rather than systemic manner, for example, via injection of the compound directly into a solid tumor.

The pharmaceutical composition of the preferred embodiments of the present invention can be formulated in a conventional manner using one or more physiologically acceptable carriers comprising excipients and auxiliaries which facilitate processing. The skilled artisan will realize that the proper formulation is dependent upon the route of administration chosen.

For injection, the pharmaceutical composition of the preferred embodiments of the present invention can be formulated in aqueous solutions, preferably in physiologically compatible buffers such as Hank's solution, Ringer's solution, or physiological saline buffer.

The pharmaceutical composition of the preferred embodiments of the present invention can comprise suitable solid or gel phase carriers or excipients. Examples of such carriers or excipients include, but are not limited to, calcium carbonate, calcium phosphate, gelatin, and polymers such as polyethylene glycols.

Dosage

For the methods of the invention, the therapeutically effective amount or dose can be estimated initially from cell culture assays. Then, the dosage can be formulated for use in animal models so as to achieve a circulating concentration range that includes the IC₅₀ as determined in cell culture. Such information can then be used to more accurately determine useful doses in humans.

Toxicity and therapeutic efficacy of the compounds described herein can be determined by standard pharmaceutical procedures in cell cultures or experimental animals, e.g., by determining the IC₅₀ and the LD₅₀. The data obtained from these cell culture assays and animal studies can be used in formulating a range of dosage for use in humans. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p.1).

Dosage amount and interval may be adjusted individually to provide plasma levels of the carrier portion containing the targeting and immune response triggering portions. These plasma levels are referred to as minimal effective concentrations (MECs). The MEC will vary for each compound but can be estimated from in vitro data. Dosages necessary to achieve the MEC will depend on individual characteristics and route of administration.

Dosage intervals can also be determined using MEC value. Compounds should be administered using a regimen that maintains plasma levels above the MEC for 10-90% of the time, preferably between 30-90% and most preferably between 50-90%.

At present, the therapeutically effective amounts of the carrier portion containing the targeting and immune response triggering portions may range from about 0.0035 g/mL to about 0.05 g/mL; preferably about 0.0035 to 0.035 g/mL or 0.005 to 0.05 g/mL.

In cases of local administration or selective uptake, the effective local concentration of the carrier portion containing the targeting and immune response triggering portions may not be related to plasma concentration. In such cases, other procedures known in the art may be employed to determine the correct dosage amount and interval.

The amount of the pharmaceutical composition of the preferred embodiments of the present invention administered will, of course, be dependent on the subject being treated, the severity of the affliction, the manner of administration, the judgment of the prescribing physician, etc.

The pharmaceutical composition can, if desired, be presented in a suitable container (e.g., a pack or dispenser device), such as an FDA approved kit, which may contain one or more unit dosage forms containing the carrier portion containing the targeting and immune response triggering portions.

In one embodiment, the pharmaceutical compositions of the preferred embodiments of the present invention may be used in conjunction with hybrid cells such as those described in co-pending U.S. application Ser. No. 09/756,293; filed Jan. 9, 2001. Such hybrid cells are useful in a variety of clinical and non-clinical applications. The hybrid cells are particularly useful in treatment regimes that invoke the immune system to treat or prevent disease. For instance, the hybrid cells can be used to treat cancer by fusing a cancer cell to an antigen presenting cell.

The compositions of the preferred embodiments of the present invention may be used to destroy bulk tumor tissue. At the same time, the hybrid cell technology can invoke a cellular immune response, thereby targeting metastatic cells.

It is also an aspect of the invention, that the pharmaceutical composition of the preferred embodiments of the present invention can be combined with other chemotherapeutic agents for the treatment of the diseases and disorders discussed above.

For instance, the pharmaceutical composition of the preferred embodiments of the present invention can be combined with alkylating agents such as fluorouracil (5-FU) alone or in further combination with leukovorin; or other alkylating agents such as, without limitation, other pyrimidine analogs such as UFT, capecitabine, gemcitabine and cytarabine, the alkyl sulfonates, e.g., busulfan, improsulfan and piposulfan; aziridines, e.g., benzodepa, carboquone, meturedepa and uredepa; ethyleneimines and methylmelamines, e.g., altretamine, triethylenemelamine, triethylenephosphoramide, triethylenethiophosphoramide and trimethylolmelamine; and the nitrogen mustards, e.g., chlorambucil, cyclophosphamide, estramustine, ifosfamide, novembrichin, prednimustine and uracil mustard; and triazines, e.g., dacarbazine.

The pharmaceutical composition of the preferred embodiments of the present invention can also be used in combination with other antimetabolite chemotherapeutic agents such as, without limitation, folic acid analogs, e.g. methotrexate and pteropterin; and the purine analogs such as mercaptopurine and thioguanine.

The pharmaceutical compositions of the preferred embodiments of the present invention can also be used in combination with natural product based chemotherapeutic agents such as, without limitation, the vinca alkaloids, e.g., vinblastin, vincristine and vindesine; the epipodophylotoxins, e.g., etoposide and teniposide; the antibiotic chemotherapeutic agents, e.g., daunorubicin, doxorubicin, epirubicin, mitomycin, dactinomycin, temozolomide, plicamycin, bleomycin; and the enzymatic chemotherapeutic agents such as L-asparaginase.

The pharmaceutical compositions of the preferred embodiments of the present invention can also be used in combination with the platinum coordination complexes (cisplatin, etc.); substituted ureas such as hydroxyurea; methylhydrazine derivatives, e.g., procarbazine; adrenocortical suppressants, e.g., mitotane, aminoglutethimide; and hormone and hormone antagonists such as the adrenocorticosteriods (e.g., prednisone), progestins (e.g., hydroxyprogesterone caproate); estrogens (e.g., diethylstilbesterol); antiestrogens such as tamoxifen; androgens, e.g., testosterone propionate; and aromatase inhibitors such as anastrozole.

Finally, the pharmaceutical compositions of the preferred embodiments of the present invention can be effective in combination with mitoxantrone or paclitaxel for the treatment of solid tumor cancers.

Having now generally described this invention, the same will be understood by reference to the following examples which are provided herein for purposes of illustration only and are not intended to be limiting unless otherwise specified.

### EXAMPLES

### Example 1

### Synthesis of NGR-gal-α-1,3gal-HSA

HSA-gal-α-1,3-gal was obtained from V-Labs, Inc. (Covington, La.) was dissolved in 0.1 M MES, 0.15 M NaCl, pH 4.7 (final concentration: 10 mg/ml) and 4 mg NGR was dissolved in 1 mL of a buffer containing 0.1 M MES, 0.15 M NaCl, pH 4.7. 500 μL NGR solution was added to 200 μl gal-α-1,3-gal-HSA solution. The NGR/gal-α-1,3-gal-HSA solution was then treated with 10 mg of EDC to give the desired NGR-gal-α-1,3-gal-HSA. Crude NGR-α-1,3-gal-HSA was purified by dialysis using a membrane with a cutoff larger than the NGR peptide, but smaller than NGR-gal-α-1,3-gal-HSA.

### Example 2

### Determination of Potential Interference, or Lack Thereof with Antibody Binding Affinity of gal-1-3-gal Incorporated into HSA

Protein G conjugated micro beads (Miltenyi Biotec) were incubated with anti-human HSA antibody at room temperature for 30 min. The beads were then divided into three groups:

Group 1: only HSA-gal-1-3-gal was added.

Group 2: an equal amount of HSA-gal-1-3-gal and HSA-fluorescein isothiocyanate (HSA-FITC) were added.

Group 3: only HSA-FITC was added.

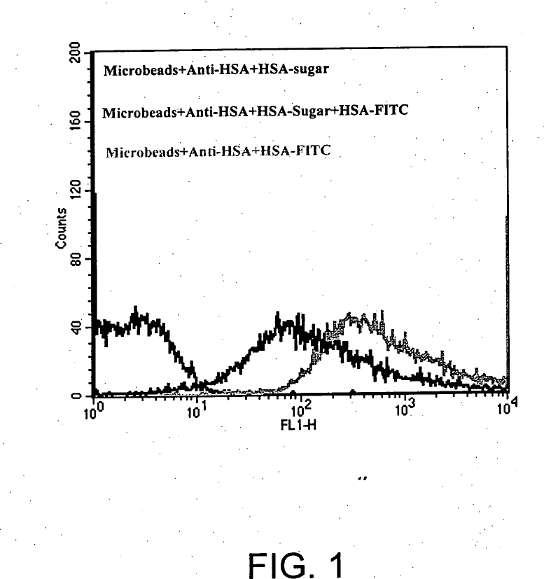

All three groups were incubated at room temperature for 30 min. After washing twice with PBS, the beads were run on the FACSCalibur flow cytometer. The result (see FIG. 1) shows that HSA-gal successfully competed with HSA-FITC, indicating that the sugar group incorporation onto HSA does not interfere with its antibody binding affinity.

### Example 3

### Induction of Cell Lysis by gal-α-1-3gal-HSA

A human natural killer lymphoma cell line, NK-92 (ATCC# CRL-2407) was used in this study. NK-92 cells are surface marker positive for CD2, CD7, CD11a, CD28, CD45, CD54 and CD56 bright. NK-92 cells were cultured in Alpha minimum essential medium with 2 mM L-glutamine adjusted to contain 1.5 g/L sodium bicarbonate with 0.2 mM inositol, 0.1 mM 2-mercaptoethanol, 0.02 mM folic acid, 100 U/ml recombinant IL-2, 75%; 12.5 house serum and 12.5% fetal bovine serum, 37° C.

1. 1.5×10⁶ NK-92 cells were labeled with ⁵¹Cr.

2. After washing, the labeled cells were evenly distributed into 21 wells of round bottom 96 well plate and assigned into 7 groups (triplicates each): 1-7. Group 1, maximum release, Group 2, nature release.

3. Group 7 was stained with rabbit anti-human CD45-Biotin (20 μl/well) for 30 min on ice and washed twice with PBS.

4. Group 7 and 6 were incubated with streptavidin (20 μl/well, 30 U/ml) for 10 min on ice and washed twice with PBS.

5. Group 7, 6 and 5 were incubated with mouse anti-human HSA-Biotin (20 μl/well, 2 mg/ml) for 10 min on ice and washed twice with PBS.

6. Group 7, 6, 5 and 4 were incubated with galα1-3gal-HSA (20 μL/well, 2 mg/ml) for 30 min on ice.

7. Group 7, 6, 5, 4, and 3 were added with 200 μl fresh human serum; Group was treated with 1× triton solution. The plate was incubated at 37° C. for 30 min.

8. After spinning at 2000 rpm for 5 min, the supernatant was transferred to counting vials containing 5 ml scintillation solution and counted on Beckman LS6500 Liquid Scintillation Counter.

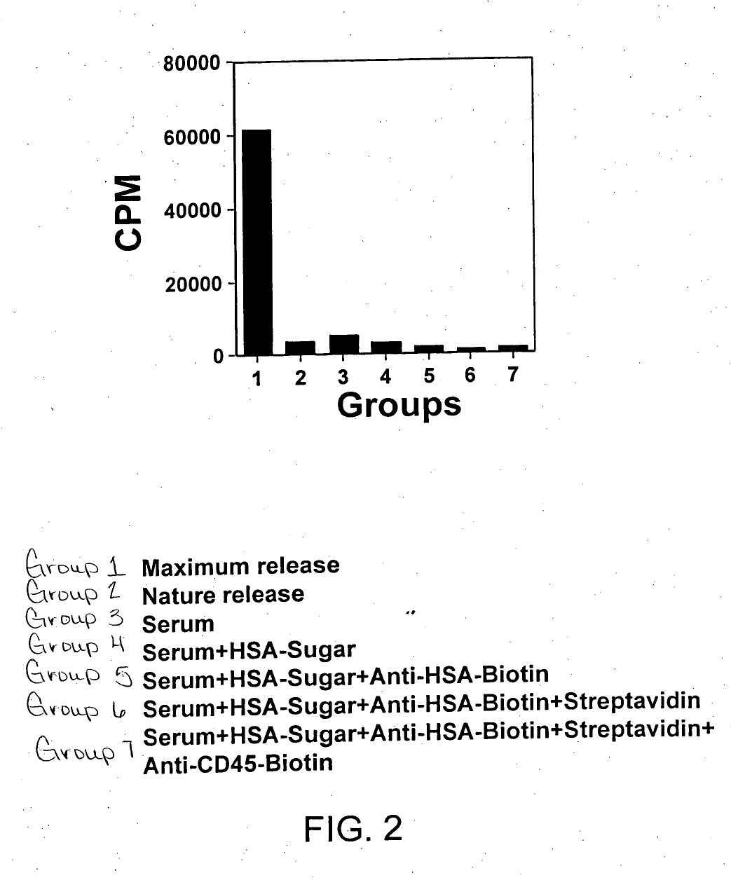

The results from these experiments are shown in FIG. 2. These results indicate that cell lysis is only observed in group 1 (maximum release) and little/no response in all the other cases (groups 2-7). The reason why no lysis was observed is because the carrier portion (HSA) bearing the triggering portion (galactose-α-1,3-galactose) is linked to the cell via an antibody. While not wishing to be bound by theory, it is believed that when an antibody is used as the targeting portion, it somehow squelches the complement response.

### Example 4

### HUVEC Cell Targeting Assay

To test whether the conjugate can bind to HUVEC cells in vitro, cells were first split into six well slide chambers and cultured overnight. On the second day, cells were first washed with PBS, FITC labeled HSA and FITC labeled HSA containing an NGR loop were added into the culture at a concentration of 1 g/ml in PBS. After one hour of incubation, the cells were washed again with PBS for three times and observed under fluorescence microscope. In this experiment, the cells which were incubated with FITC labeled HSA, wherein the HSA contained an NGR loop, fluoresced. On the other hand, the cells which were incubated with FITC labeled HSA, wherein the HSA lacked the NGR loop, did not fluoresce or fluoresced very little.

### Example 5

### In Vitro HUVEC Cell Lysis Assay

HUVEC cells were first split into six well slide chambers the same way as mentioned in the targeting assay. After washing with PBS, one ml of NGR/gal-(1,3)gal-HSA and gal-(1,3)gal-HSA at a concentration of 1 g/ml in PBS were added into the cultures and incubated for one hour. Cells were then washed again with PBS three times and 20% of freshly isolated human serum in PBS was added into the cultures and incubated for 30 minutes. Cells were again washed with PBS and then stained with a Live/Dead Viability/Cytotoxicity kit (Molecular Probe, Eugene, Oreg.) for 30 minutes according to the manufacturer suggested protocol. The stained cells were observed under a fluorescence microscope.

In this experiment, it was observed that cells that were incubated with NGR/gal-(1,3)gal-HSA either lysed or stained red. The red stain is indicative of a dead cell. On the other hand, cells that were incubated with gal-(1,3)gal-HSA stained green, thus indicating that these cells were alive. These results indicate that HUVEC cells were killed only when NGR/gal-(1,3)gal-HSA localized on the HUVEC cell surface.

### Example 6

### Targeting of Prostate Tumor Cells

MMP2 is a peptidase that is specifically expressed on prostate tumor cells. The carrier portion, in this case HSA, comprises the gal-α-1,3-gal epitope and an inhibitor of MMP2 (e.g., sodium 1-(12-hydroxy)octadecanyl sulfate; Fujita, et al. J. Nat. Prod. 65: 1936-1938 (2002).

From the foregoing description, one skilled in the art can easily ascertain the essential characteristics of this invention, and without departing from the spirit and scope thereof, can make various changes and modifications of the invention to adapt it to various usages and conditions without undue experimentation. All patents, patent applications and publications cited herein are incorporated by reference in their entirety.

1. A pharmaceutical composition comprising:
(a) a carrier portion;
(b) a targeting portion, wherein said targeting portion comprises a targeting peptide; and
(c) an immune response triggering portion, wherein said immune response triggering portion triggers a complement mediated hyperacute immune response.

2. The pharmaceutical composition of claim 1, wherein said carrier portion is human serum albumin (HSA).

3. The pharmaceutical composition of claim 1, wherein said targeting peptide comprises asparagine-glycine-arginine (NGR).

4. The pharmaceutical composition of claim 1, where said triggering portion is galactose-α-1,3-galactose.

5. A method for selectively inducing a complement mediated hyperacute immune response to a target tissue comprising treating said tissue with a pharmaceutical composition comprising a carrier portion, a targeting portion and an immune response triggering portion, wherein said targeting portion binds to cells on said tissue.

6. The method of claim 5, wherein said target tissue is the vasculature of a primary or metastatic solid tumor.

7. The method of claim 6, wherein said tumor is a lung, colorectal, bladder, prostate, breast, renal, brain, pancreatic, head, neck or an ovarian tumor.

8. The method of claim 5, wherein said carrier portion is HSA, said targeting portion is NGR and said triggering potion is gal-α-1,3-gal.

9. The method of claim 5, wherein the method of administration of said composition is intravenous.

10. A kit comprising, in a suitable container, a pharmaceutical composition comprising a carrier portion, a targeting portion and an immune response triggering portion.

11. The kit of claim 10, wherein said targeting portion and carrier portion is not an antibody or antibody fragment.

12. The kit of claim 10, wherein said targeting portion selectively binds to tumor vasculature.

13. The kit of claim 10, wherein said targeting portion is a molecule selected from the group consisting of an inhibitor, a ligand, an agonist, an antagonist, and a substrate.

14. The kit of claim 10, wherein said targeting portion comprises a targeting peptide.

15. The kit of claim 14, wherein said targeting peptide comprises asparagine-glycine-arginine (NGR).

16. The kit of claim 10, where said triggering portion triggers a complement mediated hyperacute immune response.

17. The kit of claim 16, where said triggering portion is galactose-α-1,3-galactose.

18. The kit of claim 10, wherein said carrier portion is HSA, said targeting portion is NGR and said triggering potion is galactose-α-1,3-galactose.
